(19) United States
(12) Patent Application Publication
(10) Pub. No.: US 2013/0310723 A1
(43) Pub. Date: Nov. 21, 2013

(54) HEEL PROTECTION BAND

(71) Applicant: Yueh-Hua Chiang, Taipei City (TW)

(72) Inventor: Yueh-Hua Chiang, Taipei City (TW)

(21) Appl. No.: 13/472,229

(22) Filed: May 15, 2012

(51) Int. Cl. A61F 5/00

(52) U.S. Cl. 602/27

(57) ABSTRACT

A heel protection band has an annular band body having an outer fastening portion and an inner fastening portion securely attached to each other. Thus, the band body is formed into a figure of 8 and has a first loop and a second loop. When the heel protection band is being put on, a heel of a user is mounted through the first loop first. Then the heel protection band is pulled to pass a front of an ankle of the user to allow the second loop to be mounted around the heel. Thus, the heel protection band is easily and quickly worn on a foot of the user. Even an ordinary person without any practice is capable of stably putting on the heel protection band on his own to protect his heel and ankle.

### BACKGROUND OF THE INVENTION

1. Field of the Invention

The present invention relates to a heel protection band, especially to a protection band that is mounted around a heel and an ankle of a user to hold and protect the ankle.

2. Description of the Prior Art(s)

Ligaments that connect bones to bones to form a joint and tendons that connect muscles to bones allow a human body to perform all kinds of exercises. However, when the human body exercises improperly, or when the joints and the tendons are overloaded, the joints and tendons are sprained or dislocated easily. Thus, people use all kinds of protectors to hold, support and protect the joints and the tendons. Among all the joints of a human body, an ankle is a major part that allows the human body to walk, run and jump. In order to move nimbly, the bones of the ankle are connected and protected only by ligaments and tendons. No strong muscle is disposed around the ankle. Therefore, pressure of the human body that is applied to the ankle causes the ankle to be sprained or fractured easily.

With reference to FIG. 10, a conventional way to hold the ankle is using a bandage 30. The bandage 30 is wound around the heel and the ankle in a figure of 8 to tape the ankle and the heel with a heel lock and to restrict introversion or extroversion angles of the ankle and a subtalar joint without affecting movements of the ankle. However, to wind the bandage 30 around the ankle and the heel with the heel lock requires repeated practice and skills. Therefore, an ordinary person without practice needs help from a professional to quickly and stably wind the bandage 30 around his ankle and heel and to avoid discomfort. In other words, winding the bandage 30 around the ankle and the heel with the heel lock is unable to timely protect the injured ankle. To the person who is unable to wind the bandage 30 by himself, since winding the bandage 30 takes time and cannot be done by himself, the person does not take off the bandage 30 at will even though the person feels uncomfortable. Moreover, the bandage 30 has to be discarded for hygiene reasons and therefore is not environmental friendly.

Therefore, an elastic tape is designed especially for winding around the heel and the ankle. The elastic tape is shorter than the bandage 30 and has two Velcro fasteners respectively mounted on two ends of the elastic tape. The elastic tape is wound around the heel and the ankle for just a couple rounds. Then the Velcro fasteners are attached to each other, and thus the elastic tape is stably wound on the heel and the ankle.

However, although winding the elastic tape around the heel and the ankle is slightly faster than winding the bandage 30 around the heel and the ankle, winding the elastic tape still takes time and is unable to timely protect the injured ankle. The ordinary person without practice still needs help from the professional and is unable to wind the elastic tape by himself.

To overcome the shortcomings, the present invention provides a heel protection band to mitigate or obviate the aforementioned problems.

### SUMMARY OF THE INVENTION

The main objective of the present invention is to provide a heel protection band. The heel protection band has an annular band body having an outer fastening portion and an inner fastening portion securely attached to each other. Thus, the band body is formed into a figure of 8 and has a first loop and a second loop.

When the heel protection band is being put on, a heel of a user is mounted through the first loop first. Then the heel protection band is pulled to pass a front of an ankle of the user to allow the second loop to be mounted around the heel. Thus, the heel protection band is easily and quickly worn on a foot of the user. Even an ordinary person without any practice is capable of stably putting on the heel protection band on his own to protect his heel and ankle.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a heel protection band in accordance with the present invention;

FIG. 2 is an enlarged side view of the heel protection band in FIG. 1;

FIG. 3 is an enlarged side view of a second embodiment of a heel protection band in accordance with the present invention;

FIG. 4 is an enlarged side view of a third embodiment of a heel protection band in accordance with the present invention;

FIG. 5 is an enlarged side view of a fourth embodiment of a heel protection band in accordance with the present invention;

FIGS. 6 to 8 are operational perspective views of wearing the heel protection band in FIG. 1;

FIG. 9 is another operational perspective view of the heel protection band in FIG. 1; and

FIG. 10 is an operational perspective view of a conventional bandage in accordance with the prior art.

### DETAILED DESCRIPTION OF THE PREFERRED EMBODIMENTS

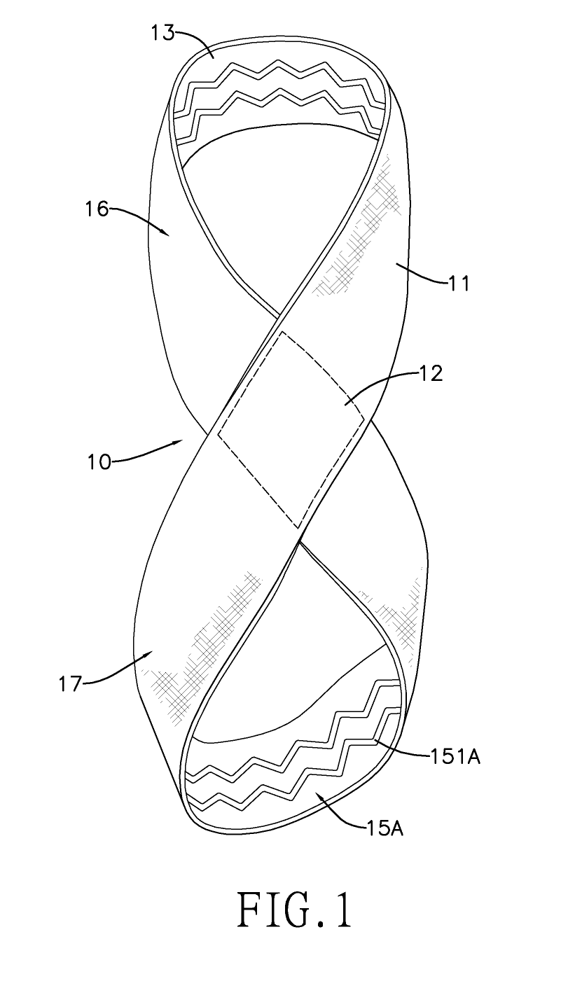

With reference to FIG. 1, a heel protection band in accordance with the present invention is elastic, may be made of elastic cloth, silicon rubber or vulcanisate, and comprises a band body 10.

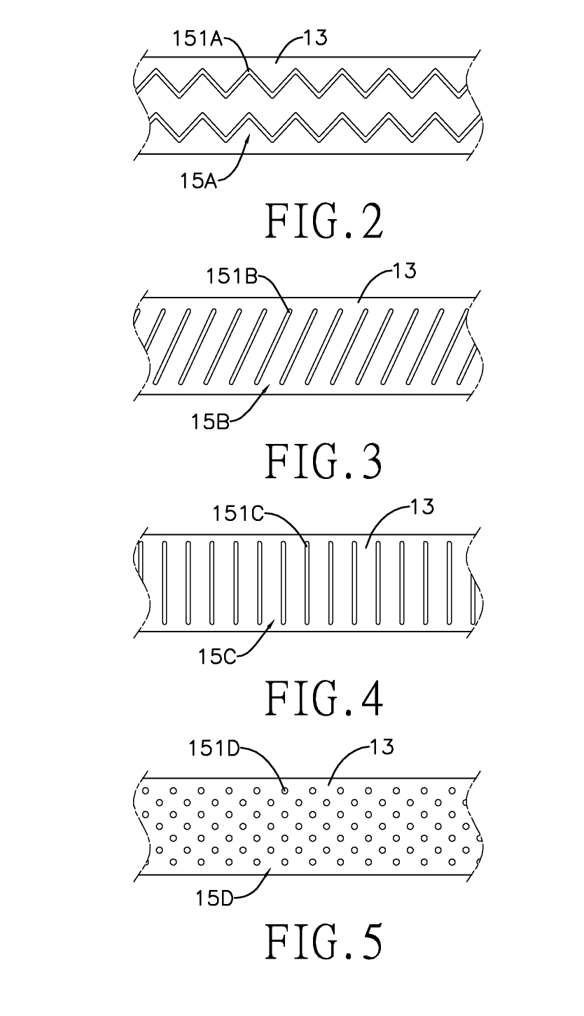

With further reference to FIGS. 2 to 5, the band body 10 is annular and has an outer surface 11, an outer fastening portion 12, an inner surface 13, an inner fastening portion and a non-slip layer 15A, 15B, 15C, 15D.

The outer fastening portion 12 is formed on the outer surface 11.

The inner fastening portion is formed on the inner surface 13, and is securely attached to the outer fastening portion 12 by method of sewing, adhesion, hot pressing and so on. Thus, the band body 10 is 8-shaped and has a first loop 16 and a second loop 17.

The non-slip layer 15A, 15B, 15C, 15D is made of non-slip materials such as silicone rubber, vulcanisate or the like, and is formed on the inner surface 13.

With reference to FIG. 2, the non-slip layer 15A has multiple non-slip strips 151A separately formed on the inner surface 13 of the band body 10. Each non-slip strip 151A is undulating and is disposed around the inner surface 13 of the band body 10.

With reference to FIG. 3, the non-slip layer 15B has multiple non-slip strips 151B separately formed on the inner surface 13 of the band body 10. Each non-slip strip 151B extends obliquely relative to a peripheral edge of the band body 10.

With reference to FIG. 4, the non-slip layer 15C has multiple non-slip strips 151C separately formed on the inner surface 13 of the band body 10. Each non-slip strip 151 C extends perpendicular to the peripheral edge of the band body 10.

With reference to FIG. 5, the non-slip layer 15D has multiple non-slip protrusions 151D separately formed on the inner surface 13 of the band body 10.

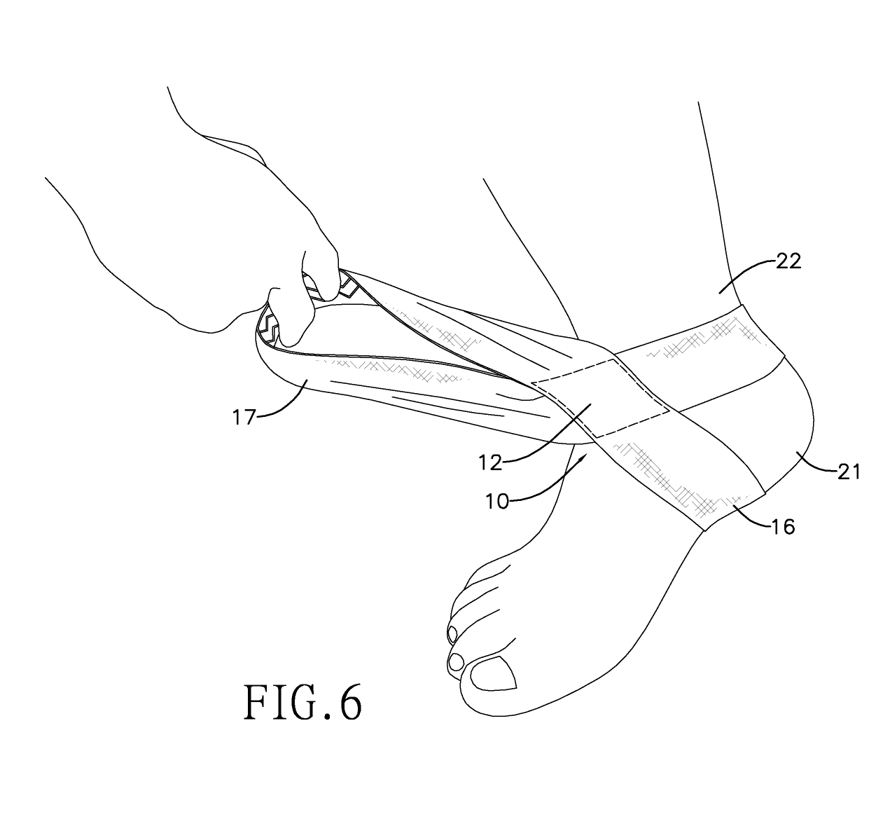

With further reference to FIG. 6, when the heel protection band is being putting on, a heel 21 of a user is mounted through the first loop 16 first. Then the heel protection band is pulled to pass a front of an ankle 22 of the user to allow the outer fastening portion 12 and the inner fastening portion of the band body 10 to press against the front of the ankle 22.

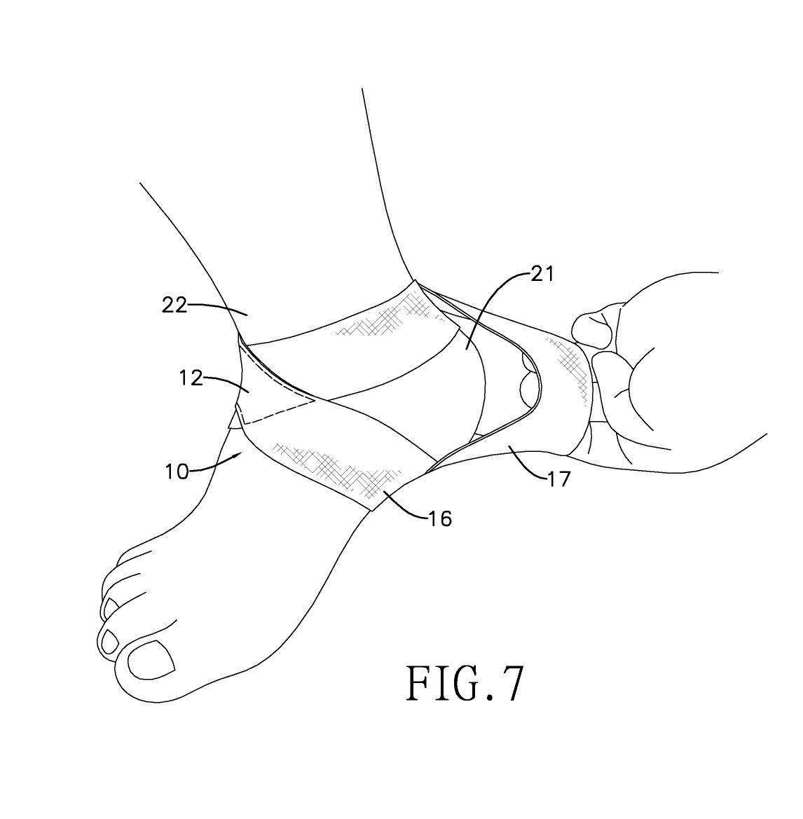

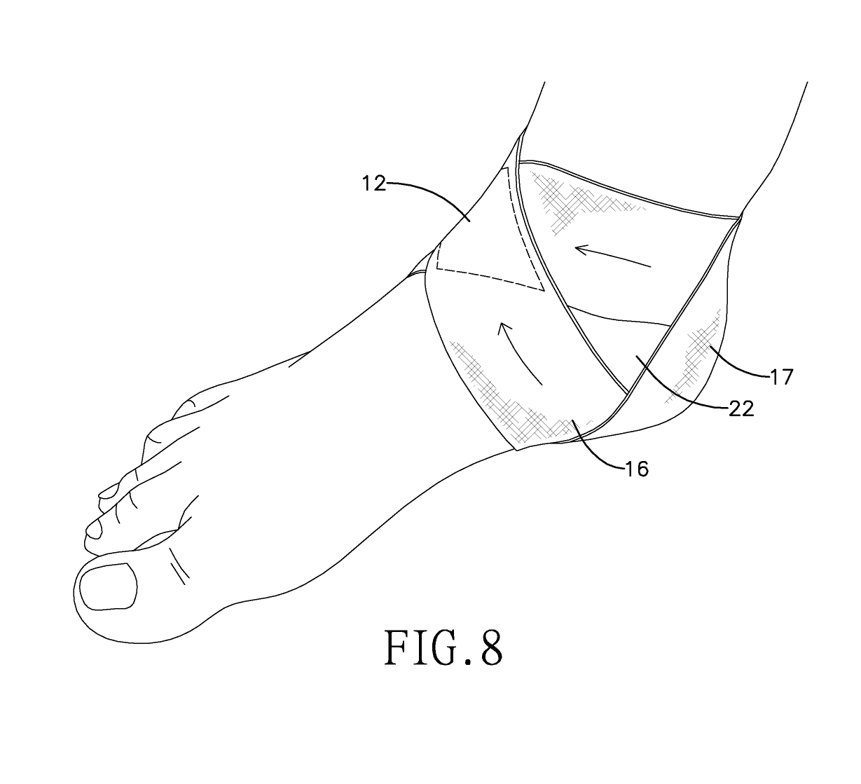

With further reference to FIGS. 7 and 8, then the second loop 17 is mounted around the heel 21. Thus, the heel protection band is easily and quickly worn on a foot of the user. The non-slip layer 15A, 15B, 15C, 15D increases friction between the heel protection band and the heel 21 of the user to prevent the heel protection band from slipping off the heel 21 of the user. Even an ordinary people without any practice is capable of stably putting on the heel protection band on his own to protect his heel 21 and ankle 22.

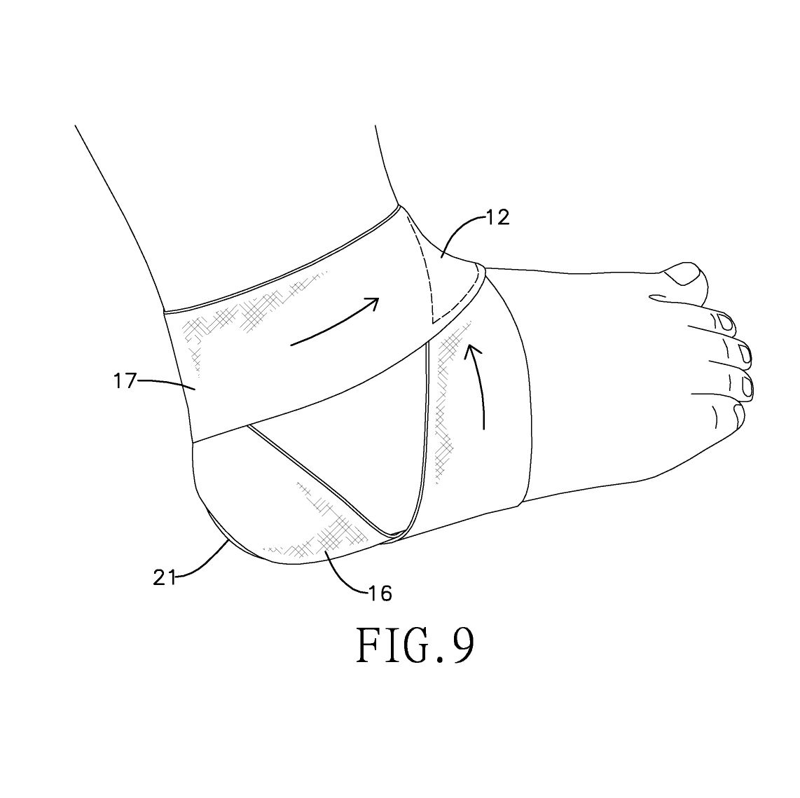

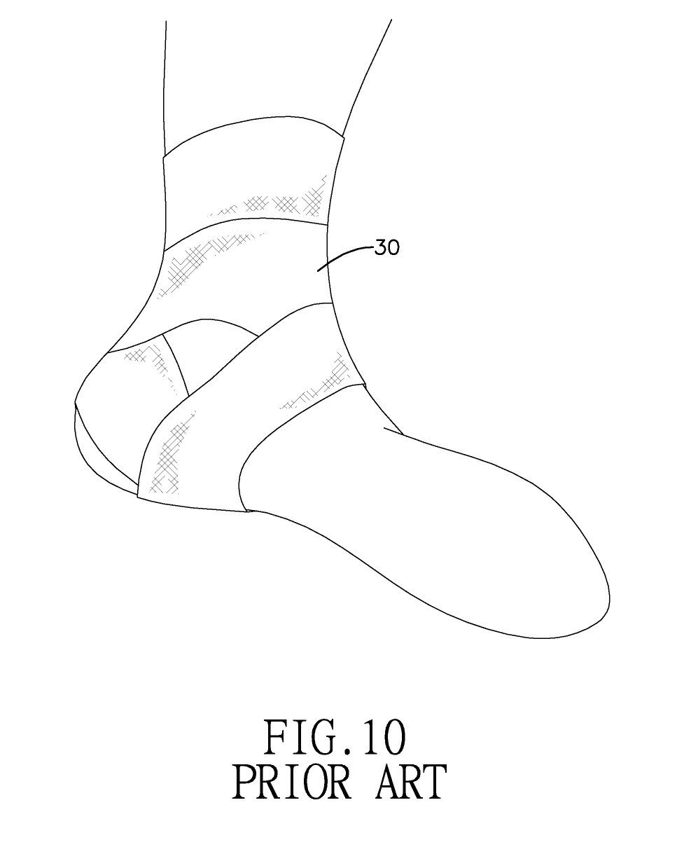

With further reference to FIGS. 8 and 9, segments of the band body 10 that are disposed adjacent to the inner fastening portion and the outer fastening portion 12 support the ankle 22 upwardly to reduce shocks applied to the ankle 22.

The first loop 16 and the second loop 17 can have the same size or have different sizes to provide the heel 21 and the ankle 22 with different protection effects.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A heel protection band being elastic and comprising a band body being annular and having:
an outer fastening portion formed on an outer surface of the band body;
an inner fastening portion formed on an inner surface of the band body and securely attached to the outer fastening portion;
thereby making the band body 8-shaped.

2. The heel protection band as claimed in claim 1, wherein the band further has a non-slip layer formed on the inner surface of the band body.

3. The heel protection band as claimed in claim 2, wherein the non-slip layer has multiple non-slip strips separately formed on the inner surface of the band body, and each non-slip strip is undulating and is disposed around the inner surface of the band body.

4. The heel protection band as claimed in claim 2, wherein the non-slip layer has multiple non-slip strips separately formed on the inner surface of the band body, and each non-slip strip extends obliquely relative to a peripheral edge of the band body.

5. The heel protection band as claimed in claim 2, wherein the non-slip layer has multiple non-slip strips separately formed on the inner surface of the band body, and each non-slip strip extends perpendicular to a peripheral edge of the band body.

6. The heel protection band as claimed in claim 2, wherein the non-slip layer has multiple non-slip protrusions separately formed on the inner surface of the band body.

7. The heel protection band as claimed in claim 1, wherein the band body has a first loop and a second loop having the same size.

8. The heel protection band as claimed in claim 2, wherein the band body has a first loop and a second loop having the same size.

9. The heel protection band as claimed in claim 1, wherein the band body has a first loop and a second loop having different sizes.

10. The heel protection band as claimed in claim 2, wherein the band body has a first loop and a second loop having different sizes.

11. The heel protection band as claimed in claim 1, wherein the inner fastening portion is securely attached to the outer fastening portion by method of sewing, adhesion or hot pressing.

12. The heel protection band as claimed in claim 2, wherein the inner fastening portion is securely attached to the outer fastening portion by method of sewing, adhesion or hot pressing.

13. The heel protection band as claimed in claim 1, wherein the non-slip layer is made of silicone rubber or vulcanisate.

14. The heel protection band as claimed in claim 2, wherein the non-slip layer is made of silicone rubber or vulcanisate.

15. The heel protection band as claimed in claim 1 made of elastic cloth, silicon rubber or vulcanisate.

16. The heel protection band as claimed in claim 2 made of elastic cloth, silicon rubber or vulcanisate.
